(19) United States
(12) Patent Application Publication
(10) Pub. No.: US 2010/0292540 A1
(43) Pub. Date: Nov. 18, 2010

(54) SURGICAL RETRACTOR AND METHOD

(76) Inventors: Christopher J. Hess, Cincinnati, OH (US); James W. Voegele, Cincinnati, OH (US); Stephan Ray Myers, Columbus, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/775,698

(22) Filed: May 7, 2010

Related U.S. Application Data
(60) Provisional application No. 61/177,431, filed on May 12, 2009.

(51) Int. Cl. A61B 1/32

(52) U.S. Cl. 600/206

(57) ABSTRACT

A retractor instrument for use in surgery having a body section and attachment tabs for securing the retractor to tissue. The retractor may have multiple body sections joined by hinges or ribs and may be rigid or flexible or some combination thereof. The retractor may comprise bioabsorbable material as well as hemostatic material. The retractor is suitable for use with clips, staples, sutures and tacks.

### RELATED APPLICATIONS

This application claims priority to U.S. Provisional Patent Application Ser. No. 61/177,431 filed on May 12, 2009.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, thoracoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery may be performed with an insufflatory fluid present within the abdominal cavity, such as carbon dioxide or saline, to provide adequate space to perform the intended surgical procedures. The insufflated cavity is generally under pressure and is sometimes referred to as being in a state of pneumoperitoneum. In thoracoscopic surgery, the rib cage maintains the working space when the lung is collapsed and therefore insufflation is not needed. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

Numerous endoscopic and minimally invasive surgical instruments have been developed that allow the surgeon to perform complex surgical procedures, whether laparoscopic, thoracoscopic, intraluminal, transluminal, or otherwise, but no one has previously made or used a device or procedure in accordance with the present invention.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts an organ retraction device.

FIG. 2 depicts a plan view of an organ retraction device.

FIG. 3 depicts an organ retraction device in an articulated position.

FIG. 4A depicts an organ retraction device.

FIG. 4B depicts a lateral to medial view of a deployed organ retraction device.

FIG. 5 depicts an anterior to posterior view of two deployed organ refraction devices.

FIG. 6 depicts a lateral to medial view of two deployed organ refraction devices.

### DETAILED DESCRIPTION

In one embodiment, a surgical retractor includes a first elongate body section having a proximal end and a distal end, and a second elongate body section has a proximal end and a distal end. The elongate body sections may be substantially rigid. The first elongate body section may be longer than the second elongate body section. An articulating link joins the proximal ends of the first and second rigid elongate body sections. The articulating link may comprise a living hinge. A first attachment tab is adjacent to the distal end of the first elongate body section, and a second attachment tab adjacent to the distal end of the second elongate body section. The articulating link and the attachment tabs may be flexible. Tissue anchors are capable of securing the attachment tabs to soft tissue. The tissue anchors may comprise sutures, clips, staples, and the like. The first attachment tab, first elongate body section, articulating link, second elongate body section, and second attachment tab may be arranged linearly as tandem segments. An organ can be retracted during surgery by obtaining a surgical retractor. The first attachment tab is secured to soft tissue using the tissue anchor. The organ is supported by with the elongate body sections. The second attachment tab is secured to soft tissue using one or more tissue anchors. In one example, the organ is a patient's liver and the first and second attachment tabs are secured to the patient's abdominal wall, and the articulating link is secured to the patient's diaphragm.

In another embodiment, a surgical retractor comprises an elongate body with two distal ends. The elongate body includes a first attachment tab segment adjacent one distal end, a second attachment tab segment adjacent the other distal end, and an elongate rigid segment between the first and second attachment tab segments. The attachment tab segments may be flexible. One of the attachment tab segments may be shorter than the elongate rigid segment. Optionally, the attachment tab segments are different lengths, and one attachment tab segment may be at least three times the length as the other attachment tab segment. The segments may be linearly arranged in tandem. Optionally, a second elongate rigid segment may be positioned between the first and second attachment tab segments, and an articulating link segment may be interposed between the first and second elongate rigid segments. Tissue anchors may be used for attaching the first and second attachment tabs to soft tissue. An organ can be refracted during surgery by obtaining a surgical retractor. The first attachment tab is secured to soft tissue using one or more tissue anchors. The organ is supported with the elongate rigid segment. The second attachment tab is secured to soft tissue using one or more tissue anchors. The steps of securing may comprise piercing the attachment tabs with one or more tissue anchors. In one example, the organ is a patient's liver, and the first attachment tab is secured to the patient's diaphragm and the second attachment tab is secured to the patient's abdominal wall. Optionally, the method may be repeated with a second surgical retractor such that the organ is supported by two surgical retractors.

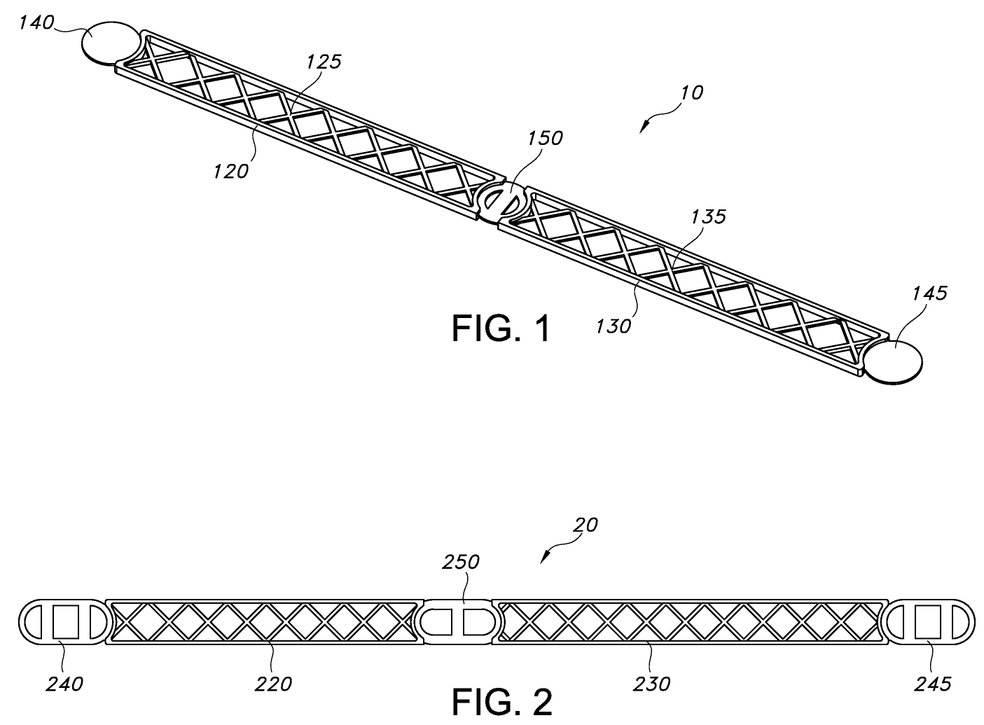

FIG. 1 depicts one example of a liver sling retractor (10) for use in laparoscopic, endoscopic, transluminal, or endoluminal surgical procedures. Retractor (10) may be constructed of a biocompatible material or combination of biocompatible materials e.g. stainless steel, polymers, or thermal plastic rubber or any other biocompatible material as is known in the art. Retractor (10) may also be constructed of absorbable material e.g. bioabsorbable magnesium alloys, polyglactin (Vicryl®), polyglycolic acid (Dexon®), or regenerated oxidized cellulose or any other bioabsorbable material, or combinations of bioabsorbable materials, as is known in the art. In one example, Rectractor (10) is constructed of a combination of polyglycolic synthetic material and regenerated oxidized cellulose. In this example, the retractor (10) provides organ retraction and hemostasis. When the retractor (10) is constructed of bioabsorbable material, its absorption profile may provide at least 80% strength for up to four hours, thereby accommodating retraction in long surgical procedures.

Retractor (10) may be constructed of a combination of materials in a braided fashion to promote flexibility. In this example, the filaments used to construct the braided retractor (10) could be made from, for example, polyester, cotton, polyamide, polyalkane, polyurethane, PET, PBT, nylon, PEEK, PE, glass fiber, metal wire, acrylic materials, and the like, or any combination or composition of the mentioned materials. They may be in the form of a monofilament or multifilament and may have a cross section of any geometric form. The materials and braiding may be selected such that the filaments are atraumatic to human tissue.

Retractor (10) in FIG. 1 comprises body sections (120, 130) disposed substantially in the same plane in a linear fashion. Retractor (10) may also be provided such that body sections (120, 130) are non-planar or curved. Sections (120,130) may be flexible, rigid, semi-rigid, or provided in any variety of stiffness, depending upon application. Refractor (10) may be provided such that body sections (120, 130) are of different stiffness. Body sections (120, 130) may be constructed such that they may be plastically or elastically deformed. Retractor (10) may be provided with a single body section or more than two body sections.

In one example, body sections (20, 30) are constructed of a mesh or lattice trusses (125, 135) to provide stiffness and/or support to the retractor (10). The mesh or lattice (125, 135) pattern may be any pattern that provides desired support and stiffness for the particular organ or structure undergoing retraction. The lattice (125) of body section (120) may be shaped differently than that of body section (130) depending upon application. Lattice (125) provides openings for viewing an organ in a retracted state thereby permitting the surgeon to view the effect of the retractor's (10) tension on an organ. For example, when retracting a large organ such as the liver, the parenchyma may bulge through the lattice (125) which may result in ischemia. In this case, another retractor or multiple retractors may be placed to reduce tension on the liver.

In another example of refractor (10), body sections (120, 130) may be solid. When body sections (20, 30) are constructed in a rigid or semi-rigid fashion, they may be formed in an arcuate cross-section to conform to the shaft of an endoscopic instrument during insertion into a body.

Body sections (120, 130) are joined at link (150). Where more than two body sections are provided, it is understood that retractor (10) may have more than one link (150). Link (150) may be constructed of the same material as body sections (120, 130). Link (150), in one example of retractor (10) is provided with openings which may promote flexibility and provide an anchoring point through which to pass suture, tacks or any other means by which to secure link (150) to tissue. Link (150), in another expression of retractor (10), may be solid and of a different thickness or material than that of body sections (120, 130). In one expression, body sections (120, 130) are arranged in an angular fashion about link (150). This angle may be anywhere from 30° to 150°.

Still referring to FIG. 1, attachment tabs (140, 145) are provided at distal ends of body sections (120, 130). Attachment tabs (140, 145) provide a pre-determined area whereby retractor (10) is attached to tissue. In one example, tabs (140, 145) are approximately 0.020″ thick permitting a clip, staple, tack or suture to pierce the tabs (140, 145) promoting attachment to tissue. In another example of retractor (10), tabs (140, 145) are constructed of a material different than the body sections (120, 130) and link (150). Tabs (140, 145) may be of different shapes and sizes to accommodate anchoring to different types of tissue and may be wider than body sections (120, 130) to accommodate multiple anchors in each tab.

Referring now to FIG. 2, another expression of an organ retractor (20) is provided. Retractor (20) comprises body sections (220, 230), link (250) and attachment tabs (240, 245). In one expression, refractor (20) is approximately 1 cm wide at its widest point. Tabs (240, 245) are formed in the shape of elongate circles and are approximately 2 cm long and provide two openings to facilitate attachment to tissue. Link (250) is approximately 2 cm long and formed in the shape of an elongate circle with two openings to facilitate attachment to tissue. Body section (220) is approximately 8 cm long measured between tab (240) and link (250). Body section (230) is approximately 6 cm long measured between link (250) and tab (245). One of skill in the art can appreciate that the body section (220, 230) can be provided in any length depending upon surgical need. Refractor (20) may be provided with a uniform cross sectional thickness or its thickness may vary as surgical application dictates. It is understood that retractor (20) may have multiple body sections as well as multiple links or may have a single body section with two tabs and no link.

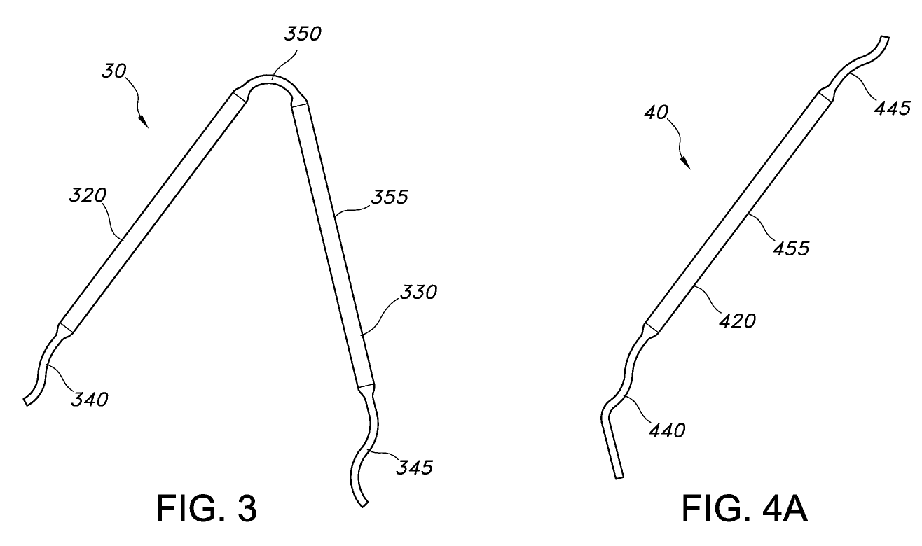

Referring now to FIG. 3, another expression of an organ retractor (30) is provided. In this expression, rigid, biocompatible dowels comprise body sections (320, 330). Sections (320, 330) may be provided in a flexible overtube (355). Overtube (355) may comprise woven materials or may be a homogeneous extrusion, or a combination thereof, or any other flexible, biocompatible material that can accommodate body sections (320, 330). Body sections (320, 330) are joined at flexible hinge (350). Hinge (350), in one expression, is formed from overtube (355). In another expression, hinge (350) is formed from flexible material joined to body sections (320, 330). In this expression, hinge (350) may be joined to body sections (320, 330) with adhesives, mechanical fasteners and the like or any other biocompatible means known to those skilled in the art.

Retractor (30) provides at least two flexible tabs (340, 345) for securing the distal ends of retractor (30) to tissue. In one expression, tabs (340, 345) are formed from overtube (355). In another expression, tabs (340, 345) are formed from flexible material joined to body sections (320, 330). In this expression, tabs (340, 345) may be joined to sections (320, 330) with adhesives, mechanical fasteners and the like or any other biocompatible means known to those skilled in the art. In one expression of retractor (30), body sections (340, 345) may be of equal length and in another expression may be of different lengths. It is understood that retractor (30) may have multiple body sections as well as multiple hinges or may have one body section with two tabs and no hinge. In another expression of retractor (30), body sections (320, 330) may be semi-rigid or flexible and may be of different cross-sections and thicknesses/diameters.

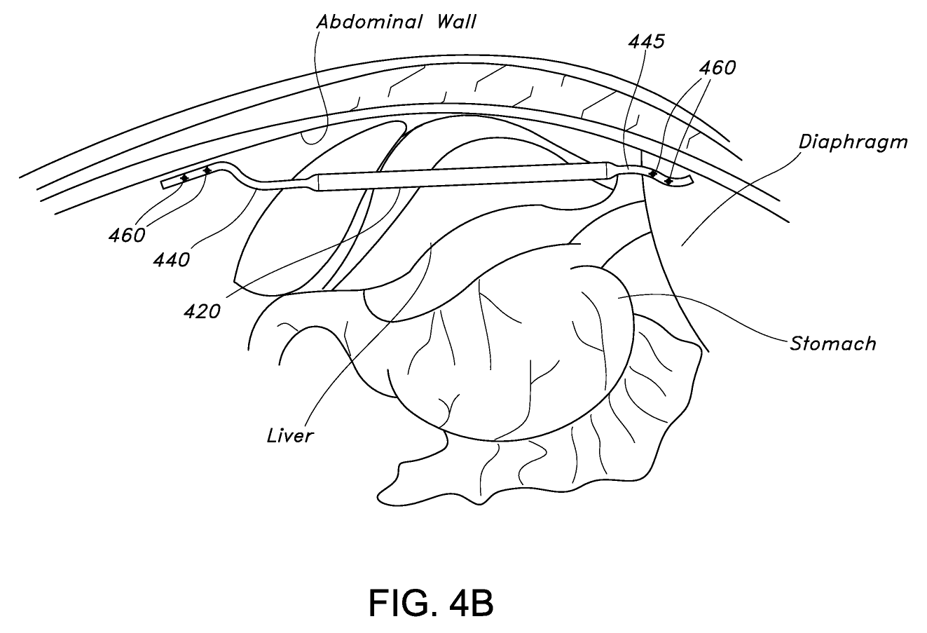

Referring now to FIG. 4A, another expression a retractor (40) comprise rigid or semi-rigid body section (420) and two flexible tabs (440, 445). The rertractor (40) constructed from a flexible overtube (455) defining the entire length of the device. The refractor (40) may have a single body section (420) defined by a dowel inserted into the overtube (455). The dowel may be shorter than the overtube (455) thus defining the body section (420), with the remainder of the overtube (455) defining the tabs (440, 445). The tabs (440, 445) may be different lengths, and optionally tab (440) may be at least three times the length of tab (445). As shown in FIG. 4B, in use the body section (420) is placed adjacent to a liver with tab (445) secured to the diaphragm with tissue anchors (460), such as sutures, tacks, clips or the like. Tab (540) is then grasped and moved adjacent to the abdominal wall. When moving tab (440) in this manner, the liver is lifted thereby exposing the lesser curvature of the stomach. Tab (440) is then secured to the abdominal wall in similar fashion to tab (545).

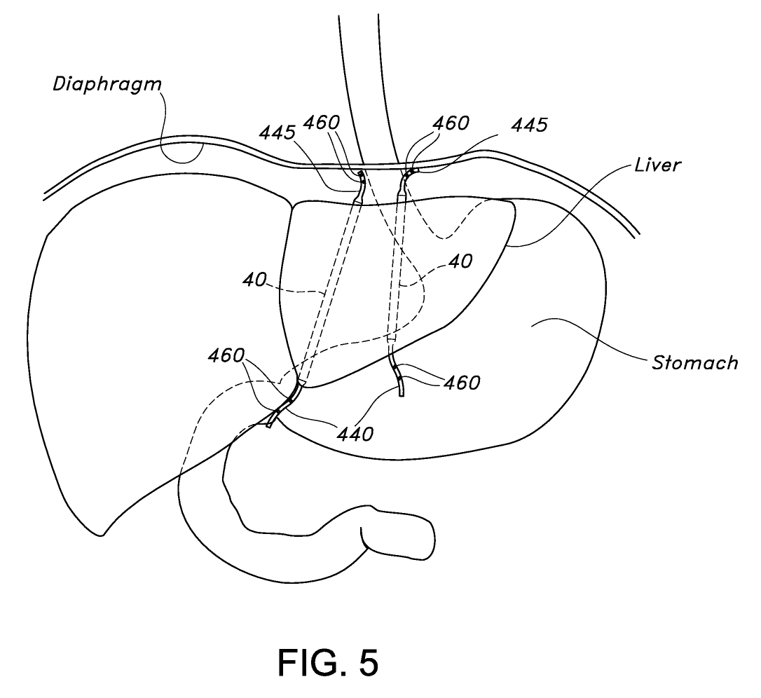

As shown in FIG. 5, more than one refractor (40) may be placed to facilitate retraction of large organs such as the liver. As shown in this example, tabs (445) are secured to the diaphragm in non-coincident locations.

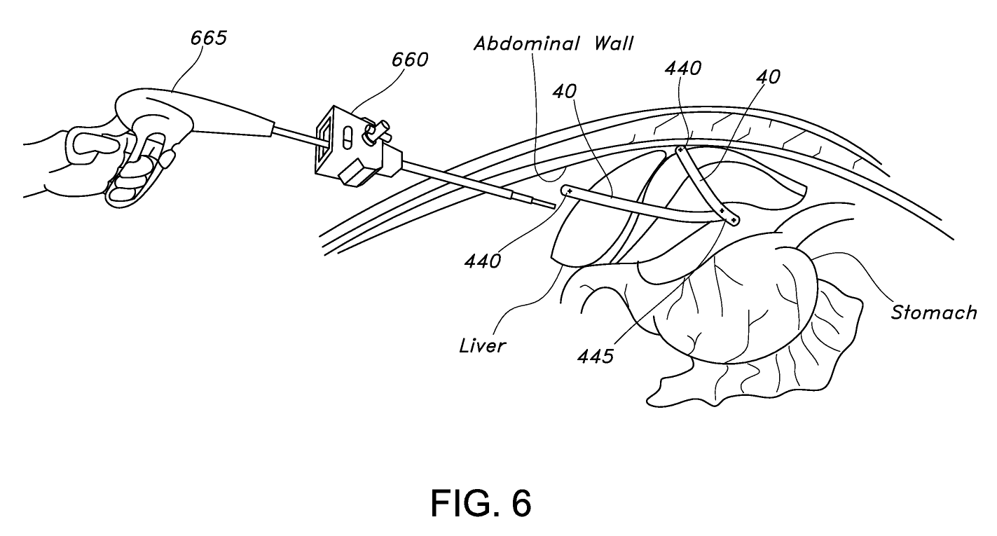

FIG. 6 depicts one expression of a retractor (40) in a fully deployed state retracting a liver. In this expression, retractor (40) was deployed through a laparoscopic trocar (660). Retractor (40) is anchored to the diaphragm and abdominal walls using a tacker (665). In this expression, the tabs (545) are secured to the diaphragm at a coincident location, resulting in a V-shaped refractor construct. If an articulating retractor is used, such as retractors (10, 20, 30), then a similar V-shaped construct may be achieved where the link (150, 250, 350) is secured to the diaphragm instead of the tab (445), and the tabs (140, 145, 240, 245, 340, 345) are secured to the abdominal wall instead of the tabs (440).

Any of the foregoing retractors may be constructed from bioabsorbable or resorbable material. In this expression, the retractors may comprise an assembly of at least two biocompatible, resorbable threads of different natures of which one is based upon regenerated oxidized cellulose and the other is a polyglycolic-type synthetic material. The threads may be twisted together before being woven or knitted into the shape of the retractor. This combination of material provides for hemostasis as well as retraction.

When hemostatic retractor may be placed on a parenchymal organ e.g. liver, lung or spleen, the organ may bleed under tension. In such a case, hemostatic retractor's cellulose elements absorb the blood through rapid dilation thereby providing hemostasis. It is understood that either all or part of retractor may comprise cellulose and polyglycol or polyglactin. For example, the tabs and link or hinge may comprise cellulose to provide hemostasis at the anchor points while substantially all of the body sections may be comprised only of polyglactin.

When used in the abdomen on organs situated cephalad e.g. liver, it may be advantageous to place a patient in the trendelenberg position, or any other head-down position. When in the trendelenberg position, the liver will rest in a manner revealing the lesser curvature of the stomach and diaphragmatic crura.

Where the expressions of the retractors set forth above comprise non-absorbable material, the retractors may be processed before surgery. First, a new or used retractor is obtained and if necessary, cleaned. The retractor can then be sterilized. In one sterilization technique, the braided device is placed in a closed and sealed container, such as a plastic or TYVEK® bag. Optionally, the retractor can be bundled in the container or kit with other components. The container and the retractor, as well as any other components, are then placed in a field of radiation that can penetrate the container, such as gamma radiation, x-rays, or high energy electrons. The radiation kills bacteria on the refractor and in the container. The sterilized refractor can then be stored in the sterile container. The sealed container keeps the retractor sterile until it is opened in the medical facility.

The retractor may be removed from its packaging in an aseptic fashion and placed in a sterile operative field. When used in endoscopic surgery, retractors may be grasped at tabs by an endoscopic babcock, grasper, dissector and the like and inserted into a trocar. Where a flexible retractor is provided, the retractor may conform to the outside of the shaft of the endoscopic insertion instrument, thereby facilitating easier insertion. Where a rigid retractor is provided, it may have an arcuate cross section to promote easier insertion. Where a rigid version of retractor is provided, a surgeon may select an appropriate operative port diameter to accommodate both the insertion instrument and the retractor. It is understood that the retractors may be introduced into a body through a trocar, thoracoport, a working channel of an endoscope or percutaneously.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the retactors may be used for organs other than the liver or in cavities other than the abdomen. The specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical retractor, comprising:
a first elongate body section having a proximal end and a distal end;
a second elongate body section having a proximal end and a distal end;
an articulating link joining the proximal ends of the first and second rigid elongate body sections; and
a first attachment tab adjacent to the distal end of the first elongate body section;
a second attachment tab adjacent to the distal end of the second elongate body section; and
tissue anchors capable of securing the attachment tabs to soft tissue.

2. The surgical retractor of claim 1, wherein the first attachment tab, first elongate body section, articulating link, second elongate body section, and second attachment tab are arranged linearly as tandem segments.

3. The surgical retractor of claim 1, wherein the articulating link comprises a living hinge.

4. The surgical retractor of claim 1, wherein the tissue anchors comprise suture.

5. The surgical retractor of claim 1, wherein the tissue anchors comprise a clip or staple.

6. The surgical retractor of claim 1, wherein the elongate body sections are substantially rigid.

7. The surgical retractor of claim 6, wherein the first elongate body section is longer than the second elongate body section.

8. The surgical retractor set forth in claim 6, wherein the articulating link and the attachment tabs are flexible.

9. A method of retracting an organ during surgery, comprising:
obtaining the surgical retractor of claim 1;
securing the first attachment tab to soft tissue using the tissue anchor;
supporting the organ with the elongate body sections;
securing the second attachment tab to soft tissue using one or more tissue anchors.

10. The method of claim 9, wherein the organ is a patient's liver and the first and second attachment tabs are secured to the patient's abdominal wall, the method further comprising attaching the articulating link to the patient's diaphragm.

11. A surgical retractor, comprising
an elongate body with two distal ends, the elongate body comprising a first attachment tab segment adjacent one distal end, a second attachment tab segment adjacent the other distal end, and an elongate rigid segment between the first and second attachment tab segments, wherein the segments are linearly arranged in tandem; and
tissue anchors for attaching the first and second attachment tabs to soft tissue.

12. The surgical retractor of claim 11, further comprising:
a second elongate rigid segment between the first and second attachment tab segments; and
an articulating link segment interposed between the first and second elongate rigid segments.

13. The surgical retractor of claim 11, wherein the attachment tab segments are flexible.

14. The surgical retractor of claim 13, wherein at least one of the attachment tab segments is shorter than the elongate rigid segment.

15. The surgical retractor of claim 14, wherein the attachment tab segments are different lengths.

16. The surgical retractor of claim 15, wherein one attachment tab segment is at least three times the length as the other attachment tab segment.

17. A surgical method for retracting an organ, comprising:
obtaining the surgical retractor of claim 11;
securing the first attachment tab to soft tissue using one or more tissue anchors;
supporting the organ with the elongate rigid segment;
securing the second attachment tab to soft tissue using one or more tissue anchors.

18. The surgical method of claim 17, wherein the steps of securing comprises piercing the attachment tabs with one or more tissue anchors.

19. The surgical method of claim 17, wherein the organ is a patient's liver, and the first attachment tab is secured to the patient's diaphragm and the second attachment tab is secured to the patient's abdominal wall.

20. The surgical method of claim 19, wherein the method is repeated with a second surgical retractor such that the organ is supported by two surgical retractors.
